(19) United States
(12) Patent Application Publication
(10) Pub. No.: US 2020/0068878 A1
(43) Pub. Date: Mar. 5, 2020

(54) METHOD FOR KILLING BED BUGS

(71) Applicant: THE CLOROX COMPANY, OAKLAND, CA (US)

(72) Inventors: Jennifer R. Gordon, PLEASANTON, CA (US); BRIAN THOMPSON, PLEASANTON, CA (US); MRUDULA SRIKANTH, PLEASANTON, CA (US)

(21) Appl. No.: 16/122,306

(22) Filed: Sep. 5, 2018

(51) Int. Cl. A01N 25/02; A01N 31/02; A61L 2/22; A01M 7/00

(52) U.S. Cl. CPC A01N 25/02; A01M 7/0046; A61L 2/22; A01N 31/02

(57) ABSTRACT

Methods for sanitizing or killing bed bugs include identifying infested surfaces for treatment, shaking a spray dispenser containing an insecticide composition, and spraying insects and/or infested surfaces from a prescribed distance (e.g., 6 to 8 inches away) for at least 3 seconds with the insecticide composition. The insecticide composition includes about 50% to about 90% alcohol (e.g., ethanol) and about 5% to about 45% water, by weight. The composition has been found to be very effective against bed bugs, and has a safer flammability rating compared to other aerosol products intended to kill bed bugs. The method may result in greater than 90% mortality against bed bugs after 96 hours, and/or greater than 80% mortality against bed bugs after 24 hours. For example, such 80%, or 90% mortality thresholds can be achieved within 1 hour.

### BACKGROUND OF THE INVENTION

### 1. The Field of the Invention

The present invention relates to methods and products that are useful in killing bed bugs, lice and similar pests.

### 2. Description of Related Art

Bed bugs (Cimex lectularius) have become an increasingly common pest. In addition, while some products claim to be effective against bed bugs, third party testing has shown that many existing products and methods have limited, if any real efficacy. For example, while some fogger and/or pyrethroid spray products are available, and the product label of such products indicates efficacy against bed bugs, various third party studies have found that such foggers are ineffective against bed bugs. In addition, the labels of some such products instruct that the product should not be applied to bedding, which is problematic, given that is where a bed bug infestation frequently occurs.

In addition, while the pyrethroid pesticide compounds included in existing compositions are a class of pesticides commonly used for indoor pest control, studies show that such compounds are not particularly effective against populations of bed bugs found in the field. Such is believed to be due to the presence of strains of bed bug populations that exhibit resistance to such pyrethroid compounds.

In addition, there is growing concern over the unintended effects that such pyrethroid and similar organic pesticides are having other beneficial pollinating insects.

As such, one article [“Cost-Effective and Money-Wasting Bed Bug Control Methods”, Wang et al., Rutgers University (2017)] suggests that there are few if any pesticides that are actually effective against bed bugs. Rather, the above article suggests that the most effective methods for dealing with a bed bug infestation are to reduce clutter and put items in plastic bins, to encase the mattress and box spring (e.g., in a vinyl encasement), to install bed bug traps, to launder or hot dry bed linens weekly, to use a heat chamber, to place small items in a freezer for 4 days, to apply steam to furniture, to remove bed bugs using a vacuum cleaner, to discard heavily infested items, and to apply repellent to pants, socks, and shoes. Such suggestions do not identify any viable pesticide treatments that will reliably kill bed bugs. For example, there do not appear to be readily available products that will reliably kill bed bugs shortly after contact.

### BRIEF SUMMARY OF THE INVENTION

The present invention is directed to methods for sanitizing a surface or article that has a bed bug present, e.g., by killing bed bugs. For simplicity, the term “bed bug” is used broadly, and encompasses similar pests, such a method may include the steps of identifying a surface with bed bugs for treatment, shaking a spray dispenser containing an insecticide composition (e.g., prior to spraying), and spraying insects (e.g., bed bugs or lice) and/or infested surfaces from a distance of at least 6 inches, for at least 3 seconds with the insecticide composition. “Infested surfaces” or a “surface being infested by bed bugs”, in the context of this invention, means any surface where bed bugs or similar pests are present it does not imply that there is a certain number of bugs or the penetration and/or the surfaces occupied by the bugs is significant. The insecticide composition includes about 50% to about 90% alcohol (e.g., ethanol) and about 5% to about 45% water by weight. The composition may include various other components, such as, but not limited to a perfume, a deodorant, a corrosion inhibitor, a pH adjustor, an antimicrobial, a solvent, or combinations thereof. Surfaces treated with such composition according to such methods exhibit greater than 90% mortality against bed bugs after 96 hours. The terms “insecticide” and “pesticide” may generally be used interchangeably herein.

Another embodiment is directed to a similar method, in which bed bugs are identified, and a spray dispenser containing the insecticide composition is shaken. Insects and/or infested surfaces are sprayed with the insecticide composition from a distance of at least 6 inches for at least 3 seconds. The insecticide composition includes about 50% to about 90% alcohol (e.g., ethanol), about 5% to about 45% water by weight, and is void of pyrethroids. Surfaces treated with such composition according to such methods exhibit greater than 90% mortality against two strains of bed bugs after 24 hours.

Another embodiment is directed to another method, in which insects and/or infested surfaces for treatment are identified, and a pressurized spray dispenser containing the insecticide composition is shaken. Insects and/or infested surfaces are sprayed with the insecticide composition from a distance of at least 6 inches for at least 3 seconds. The insecticide composition includes about 50% to about 90% alcohol (e.g., ethanol), about 5% to about 45% water, about 1% to about 25% propellant by weight, and is void of pyrethroids. The insecticide composition may have a flash point between 20° F. and 80° F. Surfaces treated with such composition according to such methods may exhibit efficacy characteristics as noted above and elsewhere herein.

The present methods advantageously provide for very effective treatment against bed bugs, similar insects, and the like, without the use of pyrethroids, neonicotinoids, or other organic pesticides. The efficacy of such compounds against bed bugs in particular is questionable, and there are concerns, e.g., regarding their unintended effects on bee and other desirable insects, e.g., which act as pollinators. Thus, at least some of the present methods use a simple but very effective composition that may simply include ethanol and water. In an embodiment, the insecticide composition may consist of these two ingredients. In other embodiments, various adjuvants may also be included, e.g., a perfume, a deodorant, a corrosion inhibitor, a pH adjustor, an antimicrobial, a solvent or co-solvent, or combinations thereof.

In addition to the simplicity of the formulation used, because the composition includes a significant fraction of water, and the concentration of ethanol or other alcohol is limited, the insecticide composition advantageously has a safer flammability rating as compared to other aerosol products intended for use against bed bugs.

Further features and advantages of the present invention will become apparent to those of ordinary skill in the art in view of the detailed description of preferred embodiments below.

### BRIEF DESCRIPTION OF THE DRAWINGS

To further clarify the above and other advantages and features of the present invention, a more particular description of the invention will be rendered by reference to specific embodiments thereof which are illustrated in the drawings located in the specification. It is appreciated that these drawings depict only typical embodiments of the invention and are therefore not to be considered limiting of its scope. The invention will be described and explained with additional specificity and detail through the use of the accompanying drawings.

FIG. 1 plots mortality over time against the Harlan strain of bed bugs, for the tested composition.

FIG. 2 plots mortality over time against the LEX8R strain of bed bugs known to be resistant to pyrethroids, for the tested composition.

### DETAILED DESCRIPTION OF THE PREFERRED EMBODIMENTS

### I. DEFINITIONS

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

The term “comprising,” which is synonymous with “including,” “containing,” or “characterized by,” is inclusive or open-ended and does not exclude additional, unrecited elements or method steps.

The term “consisting essentially of” limits the scope of a claim to the specified materials or steps “and those that do not materially affect the basic and novel characteristic(s)” of the claimed invention.

The term “consisting of” as used herein, excludes any element, step, or ingredient not specified in the claim.

It must be noted that, as used in this specification and the appended claims, the singular forms “a,” “an” and “the” include plural referents unless the content clearly dictates otherwise. Thus, for example, reference to a “surfactant” includes one, two or more such surfactants.

Numbers, percentages, ratios, or other values stated herein may include that value, and also other values that are about or approximately the stated value, as would be appreciated by one of ordinary skill in the art. As such, all values herein are understood to be modified by the term “about”. A stated value should therefore be interpreted broadly enough to encompass values that are at least close enough to the stated value to perform a desired function or achieve a desired result, and/or values that round to the stated value. The stated values include at least the variation to be expected in a typical manufacturing process, and may include values that are within 10%, within 5%, within 1%, etc. of a stated value. Furthermore, where used, the terms “substantially”, “similarly”, “about” or “approximately” represent an amount or state close to the stated amount or state that still performs a desired function or achieves a desired result. For example, the term “substantially” “about” or “approximately” may refer to an amount that is within 10% of, within 5% of, or within 1% of, a stated amount or value.

Some ranges may be disclosed herein. Additional ranges may be defined between any values disclosed herein as being exemplary of a particular parameter. All such ranges are contemplated and within the scope of the present disclosure.

Unless otherwise stated, all percentages, ratios, parts, and amounts used and described herein are by weight.

The phrase ‘free of’ or similar phrases if used herein means that the composition or article comprises 0% of the stated component, that is, the component has not been intentionally added. However, it will be appreciated that such components may incidentally form thereafter, under some circumstances, or such component may be incidentally present, e.g., as an incidental contaminant.

The phrase ‘substantially free of’ or similar phrases as used herein means that the composition or article preferably comprises 0% of the stated component, although it will be appreciated that very small concentrations may possibly be present, e.g., through incidental formation, contamination, or even by intentional addition. Such components may be present, if at all, in amounts of less than 1%, less than 0.5%, less than 0.25%, less than 0.1%, less than 0.05%, less than 0.01%, less than 0.005%, less than 0.001%, or less than 0.0001%. In some embodiments, the compositions described herein may be free or substantially free from any specific components not mentioned within this specification.

The insecticide compositions sprayed onto the bed bugs or infested surfaces as described herein may also provide sanitization, disinfection, or sterilization of the treated surfaces, As used herein, the term “sanitize” shall mean the reduction of contaminants in the inanimate environment to levels considered safe according to public health ordinance, or that reduces the bacterial or other target population by significant numbers where public health requirements have not been established. As used herein, the term “sanitize” shall mean the reduction of “target” contaminants in the inanimate environment to levels considered safe according to public health ordinance, or that reduces a “target” bacterial population by significant numbers where public health requirements have not been established. By way of example, an at least 99% reduction in bacterial population within a 24 hour time period is deemed “significant.” Greater levels of reduction (e.g., 99.9%, 99.99%, etc.) are possible, as are faster treatment times (e.g., within 10 minutes, within 5 minutes, within 3 minutes, within 2 minutes, or within 1 minute), when sanitizing.

As used herein, the term “disinfect” shall mean the elimination of many or all “target” pathogenic microorganisms on surfaces with the exception of bacterial endospores. As used herein, the term “sterilize” shall mean the complete elimination or destruction of all forms of “target” microbial life and which is authorized under the applicable regulatory laws to make legal claims as a “sterilant” or to have sterilizing properties or qualities. Some embodiments may provide for at least a 2 or more log reduction in a bacterial population within a designated time period (e.g., 10 minutes, 5 minutes, 3 minutes, 1 minute, 30 seconds, 10 seconds or the like). A 2-log reduction is equivalent to a 99% reduction, a 3-log reduction is equivalent to at least a 99.9% reduction, a 4-log reduction is equivalent to at least a 99.99% reduction, a 5-log reduction is equivalent to at least a 99.999% reduction, etc. An example of a target microbe may be Staphylococcus aureus. It will be appreciated that microefficacy can also be achieved against other target microbes, numerous examples of which will be apparent to those of skill in the art. The present methods and formulations are particularly tailored for sanitizing surfaces or articles that are infested with bed bugs, or similar pests, such as lice.

In the application, effective amounts are generally those amounts listed as the ranges or levels of ingredients in the descriptions, which follow hereto. Unless otherwise stated, amounts listed in percentage are in weight percent (based on 100 weight percent active) of the particular material present in the referenced composition, any remaining percentage being water or an aqueous carrier sufficient to account for 100% of the composition, unless otherwise noted. For very low weight percentages, the term “ppm” corresponding to parts per million on a weight/weight basis may be used, noting that 1.0% by weight corresponds to 10,000 ppm.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

### II. INTRODUCTION

Applicant has surprisingly found that a formulation including about 50% to about 90% alcohol (e.g., ethanol) by weight, and about 5 to 45% water by weight can be very effective at killing bed bugs. For example, application of such a formulation (e.g., in a pressurized, aerosol configuration) by spraying the composition onto bed bugs or a surface identified as being infested with bed bugs, from a distance of at least 6 inches, for at least 3 seconds, has been surprisingly found to kill more than 90% of bed bugs present within 96 hours. The method and composition is particularly effective, e.g., the greater than 90% mortality is typically achieved within 24 hours, within 10 hours, within 5 hours, or even within the first hour after application. Where two strains of bed bugs are present, the mortality results may include killing more than 80% of the two strains of bed bugs within 24 hours. The method and composition is particularly effective, e.g., the greater than 80% mortality is typically achieved within 10 hours, within 5 hours, or even within the first hour after application.

While some compositions are available that supposedly provide efficacy against bed bugs, the present compositions include a lower (i.e., safer) flammability rating, and in at least some embodiments, do not include any of the typical organic pesticides (e.g., pyrethroids, neonicotinoids or other complex organic, often aromatic pesticide molecules). Such organic pesticides (e.g., complex organic molecules, rather than ethanol) are increasingly being discovered to interfere with beneficial insects, such as bees and the like which are relied on as pollinators and often infective at killing bed bugs. The simplicity of ethanol as an effective pesticide against bed bugs is particularly advantageous, as it is very effective, has a relatively safe flammability rating in the formulations contemplated herein, and unintended negative side effects (e.g., killing desirable pollinators) are minimized or eliminated.

### III. EXEMPLARY COMPOSITIONS

a. Alcohol

The compositions used in the present methods include an alcohol. Ethanol is particularly preferred, although some other alcohols may also prove suitable for use. By way of example, lower alcohols (e.g., C₁-C₄) may be used. In an embodiment, the employed alcohol is not isopropyl alcohol, as such alcohol may not be effective as a pesticide against bed bugs. The same may be said for any of the lower alcohols other than ethanol (e.g., methanol, n-propyl alcohol, butanol, etc.). In a preferred embodiment, the included alcohol comprises, or may exclusively be, ethanol. The alcohol may comprise a majority of the composition, but less than 100% of the composition. For example, the composition may comprise 50% to 90%, from 50% to 75%, from 55% to 70%, or from 60% to 70% alcohol by weight.

By way of further example, if any lower alcohols other than ethanol are present, they may account for less than 40%, less than 30%, less than 25%, less than 20%, less than 15%, less than 10%, less than 5%, less than 3%, less than 2%, less than 1%, or less than 0.5% by weight of the composition. Similarly, they may account for less than 50%, less than 40%, less than 30%, less than 25%, less than 20%, less than 15%, less than 10% or less than 5% by weight of the included alcohols. For example, the composition may include no or only limited amounts of isopropyl alcohol, n-propyl alcohol, butanol (including isomers), or methanol.

b. Water

The compositions used in the present methods include a significant fraction of water. The inclusion of water, and ensuring that the alcohol content (e.g., ethanol) does not exceed 90%, 80%, or 70%, aids in ensuring that the composition has a relatively safe flammability profile. For example, the composition may have a flash point that is greater than 20° F., greater than 30° F., greater than 40° F., or greater than 50° F. For example, the composition may have a flashpoint that falls within the range from 20° F. to 80° F. By way of example, the composition may include at least 5%, at least 10%, at least 15%, at least 20%, at least 25%, or at least 30% water by weight. In an embodiment, the composition may include from 5% to 45% water by weight.

c. Solvent

In addition to the water, the composition may also include an organic solvent or cosolvent. Such an organic solvent or cosolvent may be water-miscible. Various polyols, including glycols (e.g., ethylene glycol, propylene glycol and/or dipropylene glycol), glycol ethers, and mixtures thereof may be used. Where such solvents are present, some embodiments may include them in only small amounts, for example, of not more than 20%, no more than 10%, not more than 8%, or not more than 6%, such as 5-10%, 5-8%, or 5-6% by weight.

d. Other Adjuvants

The composition may consist essentially of the ethanol, water, and optional organic solvent or cosolvent, with various optional adjuvant components that may be present, e.g., typically in very small amounts. For example, one or more of a perfume or deodorant (the term fragrance may also be interchangeable with perfume and deodorant), a corrosion inhibitor, a pH adjustor, a surfactant, or an antimicrobial may also be present. For example, such additional adjuvants may be present, if at all, in amounts of less than 5%, less than 3%, less than 2%, less than 1%, less than 0.5%, less than 0.1% or less than 0.01%.

Various surfactants and other optional adjuvants are disclosed in U.S. Pat. No. 3,929,678 to Laughlin and Heuring, U.S. Pat. No. 4,259,217 to Murphy, U.S. Pat. No. 5,776,872 to Giret et al.; U.S. Pat. No. 5,883,059 to Furman et al.; U.S. Pat. No. 5,883,062 to Addison et al.; U.S. Pat. No. 5,906,973 to Ouzounis et al.; U.S. Pat. No. 4,565,647 to Llenado, and U.S. Publication No. 2013/0028990. The above patents and applications are each herein incorporated by reference in their entirety.

The composition may have a low viscosity, e.g., such as less than 1000 cps, 1 cps to 100 cps, 1 cps to 10 cps, or 1 cps to 5 cps. In an embodiment, the viscosity may be similar to that of water (e.g., about 1 cps), facilitating spraying the composition from a pressurized aerosol container.

e. Pyrethroids and Other Complex Organic Pesticides

While in at least some embodiments, no pyrethroids or other complex organic pesticide compounds are included, such organic pesticides could be included. Pyrethroids refer to organic compounds similar to natural pyrethrins that are produced by the flowers of pyrethrums (Chrysanthemum cinerariaefolium and C. coccineum). Pyrethroids constitute the majority of commercial household insecticides.

Pyrethroids are axonic excitotoxins, the toxic effects of which are mediated through preventing the closure of the voltage-gated sodium channels in axonal membranes of target insects, as described in the Wikipedia entry on Pyrethroids. Many such pyrethroids include an ester group. Many also include a benzene ring. It will be apparent they are complex organic molecules. Many such compounds have names that include or end in -thrin. Non-limiting examples of pyrethroids include permethrin, allethrin, bifenthrin, cyfluthrin, cypermethrin, cyphenothrin, deltamethrin, esfenvalerate, etofenprox, fenpropathrin, fenvalerate, flucythrinate, flumethrin, imiprothrin, lambda-cyhalothrin, metofluthrin, phenothrin (e.g., D-Cis trans phenothrin), prallethrin, resmethrin, silafluofen, sumithrin, tau-fluvalinate, tefluthrin, tetramethrin, tralomethrin, transfluthrin, and combinations thereof.

There are various other classes of complex organic pesticides, such as neonicotinoids. Neonicotinoids are a class of neuro-active pesticides that are derivatives and/or share chemical similarities to nicotine. For example, such compounds often include a nitrogen-substituted benzene ring, and/or a nitrogen-substituted cylcopentane ring. Many such compounds have names that include or end in -prid, -din, -pyram, or -oxam. Examples of neonicotinoids include acetamiprid, cl othi ani din, imidacloprid, nitenpyram, nithiazine, thiacloprid, thi am ethoxam, and combinations thereof.

Combinations of different complex organic pesticides are sometimes used in an effort to improve efficacy against resistant strains of pests. For example, combinations of pyrethroids, neonicotinoids, or other classes of pesticides. As described above, there are concerns with use of such complex organic pesticides, such that in at least some embodiments, no such complex organic pesticides are present. In another embodiment, one or more such complex organic pesticides may be included in combination with the ethanol (or other alcohol).

f. Propellant

As the composition may typically be dispensed from a pressurized container as an aerosol, the composition or container may further comprise a propellant. In an embodiment, the spray dispensing device used to dispense the composition may be free of hydrocarbon propellants. The elimination of the use of any hydrocarbon propellants reduces or eliminates the detrimental environmental effects associated with use of such propellants in existing dispensing systems. For example, a “bag-on-valve” or “bag-in-bottle” type dispensing system may be used, as will be appreciated by those of skill in the art. Additional details of such dispensing systems are disclosed in Applicant's U.S. Publication No. 2018/0072486, herein incorporated by reference in its entirety. Where such bag-on-valve or bag-in-bottle configurations are used, the pressurized composition used to dispense the composition may be air, nitrogen, carbon dioxide, or other relatively environmentally inert pressurized gas. In other embodiments, a hydrocarbon propellant, such isopentane, propane, isobutane, pentane, butane or the like may be used. Preferably no fluorocarbon propellants are used.

Where the propellant is included with the composition (e.g., not in a “bag-on-valve or “bag-in-bottle” configuration), the propellant may comprise from about 1% to about 25%, from about 1% to 20%, or from about 2% to about 10% by weight of the composition. Where a “bag-on-valve or “bag-in-bottle” configuration is used, the propellant may be separate from the composition.

The method may involve spraying the composition onto a desired surface that is infested with bed bugs, for a minimum period of time, e.g., at least 3 seconds. For example, when the composition is packaged within a pressurized container, the container may be operated to spray or otherwise dispense the composition for 3 seconds, 4 seconds, 5 seconds, or the like. Dispensing times may range from 3 seconds to 30 seconds, 3 seconds to 15 seconds, or 3 seconds to 10 seconds, for example.

A 5 second application may deliver, for example, 4 g to 5 g of composition over that application time. As such, another way to characterize application of the composition may be by weight. For example, the method may involve applying at least 2 g, at least 3 g, or at least 4 g of the composition to a desired infested surface. For example, from 2 g to 10 g, from 2 g to 7 g, or from 3 g to 7 g of the composition may be applied to a given infested surface to achieve sanitization thereof.

The composition may typically be sprayed or otherwise applied at a distance of at least 6 inches, such as from 6 to 12 inches, or 6 to 8 inches from the bed bug infested surface being treated.

The compositions as described herein can advantageously be applied to infested soft or hard surfaces without causing surface damage or staining. For example, while some existing compositions intended to eradicate bed bugs may specifically instruct that the composition should not be applied to bedding, e.g., because of risks or concerns over flammability, staining, or other damage, the present compositions largely if not completely eliminate such problems.

### IV. EXAMPLES

### Example 1

The efficacy of a given composition was tested against bed bugs (Cimex lectularius) according to applicable EPA product performance test guidelines (e.g., OCSPP 810-3900).

The tested composition was packaged within a pressurized spray container, and included 66.34% ethanol, 5.31% dipropylene glycol, and 23% water. The balance of the composition (about 5.35% thereof) included a propane propellant, d-limonene as a fragrance or perfume, and sodium nitrite as a corrosion inhibitor.

The composition was evaluated for direct contact application against two strains of bed bugs (Cimex lectularius) according to applicable EPA protocols. Both laboratory susceptible and field collected (≤F₂ generation) bed bugs were provided in 8 oz. plastic containers with mattress ticking to absorb excess liquid. The lab susceptible strain was “Harlan”. The field strain was “Arizona”, collected from single and multi-family dwellings in Arizona. The test composition was applied as an aerosol spray at 6-8 inches from the bed bugs, as a 5-second application to the bed bugs on top of the substrate, with 5 replicates of 10-11 adult bed bugs in each replicate (1:1 sex ratio). The amount of composition dispensed ranged from 4.78 g to 4.85 g. Knockdown was assessed at 30 seconds, and mortality was assessed at 30 seconds, 1, 5, 15, 30, 45, and 60 minutes, and at 2, 4 ,6, 24, 48, 72 and 96 hours after treatment. Knockdown is defined as a bed bug that is incapable of coordinated movement to right itself following exposure to the composition. Knockdown typically precedes mortality. The bed bugs were transferred to clean test containers with filter paper in the bottom 1-hour after treatment. Untreated control replicates were set up, transferred, and scored in the same manner as the treated groups.

The composition provided rapid mortality, 100% at 5 minutes after treatment, against both the laboratory susceptible and field collected strains of bed bugs using a direct aerosol application method, as shown in Table 1. Table 1 reports the mortality percentage at each given interval, up to 5 minutes. Intervals after 5 minutes are not included in Table 1 as mortality was already 100% at 5 minutes). The values in parenthesis report the standard deviation.

TABLE 1

| Strain | | 30 sec. | 1 min. | 5 min. |
|---|---|---|---|---|
| Harlan | Control | 0.0 | 0.0 | 0.0 |
| | | (0) | (0) | (0) |
| | Tested | 25.0 | 86.5 | 100 |
| | Composition | (8.5) | (5.0) | (0) |
| Arizona | Control | 0.0 | 0.0 | 0.0 |
| | | (0) | (0) | (0) |
| | Tested | 32.0 | 84.0 | 100 |
| | Composition | (5.8) | (6.8) | (0) |

### Example 2

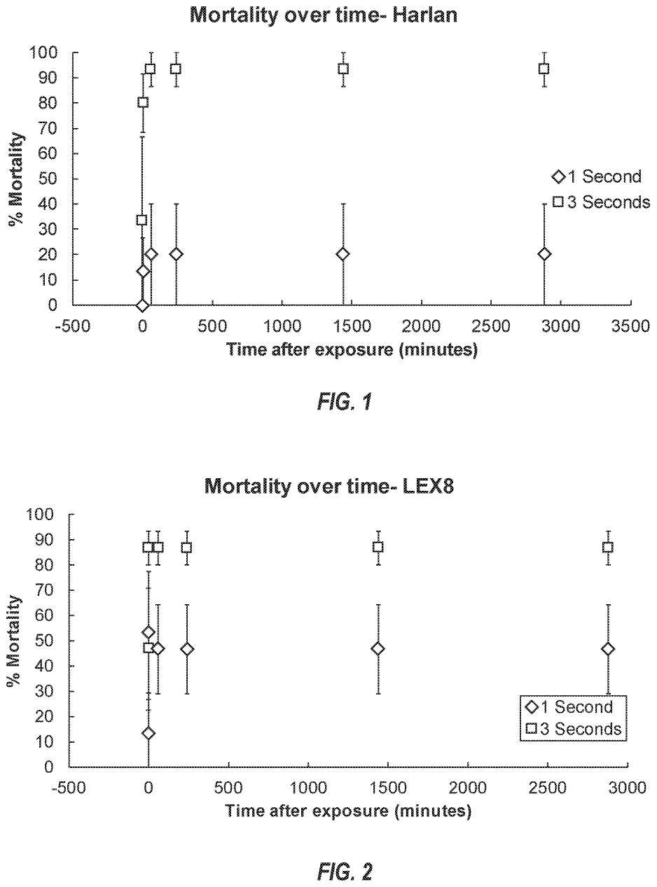

The same composition as in Example 1 was tested under similar conditions as described in Example 1, but where the 2 bed bug strains were Harlan and LEX8R (a strain known to be resistant to pyrethroids), and the application time was 3-seconds rather than 5 seconds. After 24 hours, the mortality against the Harlan strain was 93.3% (±3.3%), and the mortality against the LEX8R strain was 86.7% (±6.7%). FIGS. 1 and 2 plot the results against Harlan and LEX8R, respectively, over the full 48 hour test period.

### Example 3

The same composition as in Examples 1 and 2 was tested under conditions and against bed bug strains identical to those of Example 2, but for a 1-second application time. After 24 hours, the mortality against the Harlan strain was about 20%, and the mortality against the LEX8R strain was about 45%. FIGS. 1 and 2 plot the results against Harlan and LEX8R, respectively, over the full 48 hour test period.

Without departing from the spirit and scope of the invention, one of ordinary skill can make various changes and modifications to the invention to adapt it to various usages and conditions. As such, these changes and modifications are properly, equitably, and intended to be, within the full range of equivalence of the following claims.

1. A method for sanitizing or killing bed bugs comprising:
(a) identifing infested surfaces for treatment;
(b) shaking a spray dispenser containing an insecticide composition prior to spraying;
(c) spraying at least one of insects or infested surfaces for at least 3 seconds with the insecticide composition, wherein the insecticide composition comprises:
(i) about 50% to about 90% alcohol by weight; and
(ii) about 5% to 45% water by weight;
(d) wherein treated surfaces have a greater than 90% mortality rate against bed bugs after 96 hours.

2. The method of claim 1, wherein the insecticide composition further comprises a propellant.

3. The method of claim 1, wherein the insecticide composition further comprises a deodorant.

4. The method of claim 1, wherein the insecticide composition further comprises a perfume.

5. The method of claim 1, wherein the insecticide composition further comprises a corrosion inhibitor.

6. The method of claim 1, wherein the insecticide composition comprises from about 50% to about 75% alcohol by weight.

7. The method of claim 1, wherein the alcohol comprises ethanol.

8. The method of claim 1, wherein the insecticide composition further comprises at least one pyrethroid.

9. The method of claim 1, wherein the treated surface has at least an 80% mortality rate against two strains of bed bugs after 24 hours.

10. The method of claim 1, wherein composition is sprayed onto insects or infested surfaces from a distance of about 6 to about 8 inches.

11. A method for sanitizing and decontaminating surfaces comprising:
(a) identifing infested surfaces for treatment;
(c) shaking a spray dispenser containing an insecticide composition;
(c) spraying at least one of insects or infested surfaces from a distance of at least about 6 inches, for at least 3 seconds with the insecticide composition, wherein the insecticide composition comprises:
(i) about 50% to about 90% alcohol by weight; and
(ii) about 5% to 45% water by weight;
(iii) wherein the composition is void of pyrethroids;
(d) wherein treated surfaces have a greater than 80% mortality rate against two strains of bed bugs after 24 hours.

12. The method of claim 11, wherein the insecticide composition further comprises at least one of a propellant, a deodorant, a perfume, or a corrosion inhibitor.

13. The method of claim 11, wherein the alcohol comprises ethanol.

14. The method of claim 13, wherein the insecticide composition comprises from about 50% to about 75% ethanol by weight.

15. A method for sanitizing and decontaminating surfaces comprising:
(a) identifing infested surfaces for treatment;
(d) shaking a pressurized spray dispenser containing an insecticide composition;
(c) spraying insects and infested surfaces from a distance of at least about 6 inches, for at least 3 seconds with the insecticide composition, wherein the insecticide composition comprises:
(i) about 50% to about 90% ethanol by weight;
(ii) about 5% to about 45% water by weight;
(iii) about 1% to about 25% propellant by weight; and
(iv) wherein the composition is void of pyrethroids;
(d) wherein the insecticide composition in the pressurized spray dispenser has a flash point between 20° F. and 80° F.

16. The method of claim 15, wherein the insecticide composition comprises from about 50% to about 75% ethanol by weight.

17. The method of claim 15, wherein the insecticide composition further comprises a deodorant.

18. The method of claim 15, wherein the insecticide composition is applied to infested soft or hard surfaces without causing surface damage or staining.

19. The method of claim 15, wherein the insecticide composition consists of:
(i) about 50% to about 90% ethanol by weight;
(ii) about 5% to about 45% water by weight;
(iii) about 1% to about 25% propellant by weight; and
(iv) optionally, one or more adjuncts selected from a perfume, a deodorant, a corrosion inhibitor, a pH adjustor, an antimicrobial, or a combination thereof.

20. The method of claim 15, wherein the composition further comprises a glycol cosolvent, a perfume or deodorant, and a corrosion inhibitor.
